SEQUENCE LISTING (3 sequences)

SEQ ID NO: 1 - DNA, 12 bp, Artificial Sequence
  base sequence
acgtacgtacgt

SEQ ID NO: 2 - DNA, 18 bp, Artificial Sequence
  base sequence
acgtaaacgtaaacgtaa

SEQ ID NO: 3 - DNA, 96 bp, Artificial Sequence
  full-length DNA sequence
acgtaccacatcagtcacgtagtcagtgctgatgtgctgacaacctacgttcgtgatgtgctgactacgttcacgtagtcagtcatgctacactac

(19) United States
(12) Patent Application Publication
(10) Pub. No.: US 2024/0419325 A1
(43) Pub. Date: Dec. 19, 2024

(54) Method for Generating a DNA Storage Encoding/Decoding rule, and Method for DNA Storage Encoding/Decoding

(71) Applicant: BGI SHENZHEN, Shenzhen, Guangdong (CN)

(72) Inventors: Haoling ZHANG, Shenzhen, Guangdong (CN); Zhi PING, Shenzhen, Guangdong (CN); Shihong CHEN, Shenzhen, Guangdong (CN); Yue SHEN, Shenzhen, Guangdong (CN)

(21) Appl. No.: 18/007,752

(22) PCT Filed: Jun. 3, 2020
(86) PCT No.: PCT/CN2020/094192

§ 371 Date: Dec. 2, 2022

(51) Int. Cl. G06F 3/06

(52) U.S. Cl. CPC G06F 3/0608; G06F 3/0655; G06F 3/0673

(57) ABSTRACT

A method for generating a DNA storage encoding/decoding rule, and a method for DNA storage encoding/decoding. The method for generating the DNA storage encoding/decoding rule includes: setting a sliding window for the DNA storage encoding/decoding rule; screening out, from a full set of sequences, a set of qualified sequences complying with a limiting condition; connecting the sequences in the set of qualified sequences by means of a directed graph; deleting, in the directed graph, nodes of which the number of out-degree is less than a set threshold for the number of out-degree; deleting excess out-degree of each node in the directed graph; and acquiring an algorithm chart, which includes the DNA storage encoding/decoding rule.

### SEQUENCE LISTING

The instant application contains a Sequence Listing that was submitted electronically in ASCII format and is hereby incorporated by reference in its entirety. Said ASCII copy named PN185848 SEQ_LIST_ST25.txt, is created on Aug. 14, 2023 and is 1,018 bytes in size. The sequence listing contains 3 sequences added from the specification of the PCT application and includes no new matter.

### TECHNICAL FIELD

The present disclosure relates to the technical field of information storage, in particular to a method and device for generating a DNA storage encoding/decoding rule, and a method and device for DNA storage encoding/decoding.

### BACKGROUND ART

With the development of modern technologies, especially the Internet, global data is growing exponentially. The ever-increasing amount of data proposes higher and higher demands on storage technologies. The traditional storage technologies, such as tape and optical disk storage, are increasingly unable to meet existing data demands due to the limited storage density and time. In recent years, the development of a DNA storage technology provides a new way to solve these problems. Compared with traditional storage media, DNA as a medium for information storage has the advantages of a long storage time (it can reach more than thousands of years, which is more than 100 times greater than that of the existing tape and optical disk media), a high storage density (it reaches about 109 Gb/mm3, which is more than ten million times greater than that of the existing tape and optical disk media) and good storage security and the like.

As shown in FIG. 1, DNA storage usually includes the following steps: 1) encoding: converting a binary 0/1 code of computer information into A/T/C/G DNA sequence information; 2) synthesizing: using a DNA synthesis technology to synthesize a corresponding DNA sequence, and preserving an acquired DNA molecule in a medium in vitro or a living cell; 3) sequencing: using a sequencing technology to read the DNA sequence of the stored DNA molecule; and 4) decoding: using a mode in the step 1 corresponding to the encoding process, converting the DNA sequence obtained by sequencing into the binary 0/1 code, and further converting into the computer information. In order to achieve the efficient DNA data storage, a technology needs to be developed for the above step process. Herein, the encoding and decoding technologies involved in the step 1 and the step 4 are the most critical technologies for the DNA data storage. The most critical parts of this technology are as follows: 1) how to maximize the density of the 0/1 binary information encoded by DNA, the increase in DNA storage density is crucial to save the cost of DNA synthesis for storing information in the step 2; and 2) while the 0/1 binary information is converted into an A/T/C/G sequence, situations of single base repeat, high GC and high AT between sequences are avoided to the greatest extent. Generally speaking, the presence of continuous single base repeat, high GC or high AT in the DNA sequence can cause difficulties in reading sequence information during the sequencing process. The way of converting the binary 0/1 data and the A/T/C/G DNA sequence directly determines the difficulty of interpreting the DNA sequence during the sequencing process, thereby the fidelity of the data during the reading process is determined.

At present, third-generation sequencing, namely single-molecule sequencing, becomes more and more popular in the sequencing industry. PacBio's SMRT and ONTs Nanopore technology are the mature members in the third-generation sequencing technology. Although the third-generation sequencing technology has the advantage of a fast sequencing speed compared with a second-generation high-throughput sequencing technology, its high error rate is one of important bottlenecks that inhibit its wide application. By the artificial design of the sequence, such as controlling its GC content, and removing a special motif, etc., the accuracy of the third-generation sequencing can be greatly improved. To achieve instant and fast reading of the DNA storage, the third-generation sequencing must be used. Therefore, it is necessary to design an encoding technology that can satisfy arbitrary limiting conditions of the DNA sequence.

Existing DNA storage encoding/decoding algorithms, classical methods include a variety of algorithms proposed by Church, Goldman, Grass, Erlich et al. The existing algorithms focus on increasing the encoding density, or try to avoid the extreme GC content, or try to avoid the continuous single base repeat and the like. However, these algorithms cannot completely avoid the situations of the extreme GC or the special motif due to their fixed rules. While the third-generation sequencing is used for sequencing and decoding, a large amount of computing time is required for error correction.

### SUMMARY

A purpose of the present disclosure is to provide a method and device for generating a DNA storage encoding/decoding rule and a method and device for DNA storage encoding/decoding, which can solve a problem of extreme GC or special motif that cannot be completely avoided by the fixed rules known by the inventor.

According to a first aspect of the present disclosure, the present disclosure provides a method for generating a DNA storage encoding/decoding rule, includes:
(1) a sliding window (n, k) is set for the DNA storage encoding/decoding rule, wherein n represents a length of the sliding window, and k represents a length of a base character for each sliding, and wherein n and k are positive integers, n≥k;
(2) based on the length n of the sliding window, a full set of sequences are obtained, wherein the full set of the sequences is a set of all base sequences formed by random combination of all base possibilities of each base position within the length of the sliding window, a set of qualified sequences complying with the above limiting condition in the above full set of the sequences is screened out by using a limiting condition, wherein the above limiting condition is set based on a sequence feature in the above full set of the sequences;
(3) the sequences are connected in the above set of the qualified sequences by means of a directed graph, wherein each node in the directed graph represents each sequence;
(4) in the above directed graph, nodes of which a number of out-degree is less than a set limiting value for the number of out-degree are deleted;
(5) an excess out-degree of each node in the above directed graph is deleted, wherein the excess out-degree is an out-degree exceeds the set limiting value for the number of the out-degree; and
(6) an algorithm chart is obtained, wherein the algorithm chart comprises the DNA storage encoding/decoding rule.

Optionally, the above limiting condition includes at least one of a GC base content, a single base repeat, a simple sequence repeat, a palindromic sequence repeat, a complementary palindromic sequence repeat and a special sequence elimination.

Optionally, the above limiting condition includes at least one of the followings:
the GC base content is 40%-60%, the single base repeat is no more than 3 consecutive identical bases, the simple sequence repeat is not less than 4 bases, the palindromic sequence repeat is not less than 4 bases, the complementary palindromic sequence repeat is not less than 4 bases, and the special sequence elimination is to eliminate a sequence containing AGA, GAG, CTC, and TCT.

Optionally, the above set limiting value for the number of the out-degree is a number of the out-degree required by an encoding efficiency.

Optionally, the above encoding efficiency is e, and while eϵ(0,2], a limiting value for the number of the out-degree on a k-th (k=e/(2−e)) layer of each node is 2\hat{ }(e/(2−e)).

Optionally, while the above encoding efficiency is 1, the limiting value for the number of the out-degree of each node is 2.

Optionally, the above step of the excess out-degree of each node in the above directed graph is deleted includes: if a total number of the out-degree of the above node exceeds the set limiting value for the number of the out-degree, bases of the above node are output in a reverse order, and an out-degree pointing to a corresponding base sequentially is deleted according to a base order output in the reverse order.

Optionally, after the step (4), the above method further includes:
(4′) in the above directed graph, nodes of which the number of in-degree is 0 are deleted.

Optionally, the above method further includes:
after the step (4′) is executed, the step (4) is returned again, and the steps (4)-(4′) are executed circularly until a number of the out-degree of all nodes in the above directed graph is greater than the set limiting value for the number of the out-degree, and there is no node of which the number of the in-degree is 0 in the above directed graph.

Optionally, between the step (4) and the step (4′), the above method further includes:
(4″) the excess out-degree of each node in the above directed graph is deleted, wherein the excess out-degree is the out-degree exceeds the limiting value for the number of the out-degree.

Optionally, the above method further includes:
after the step (4′) is deleted, the step (3) is returned again, and the steps (3)-(4)-(4″)-(4′) are executed circularly until the number of the out-degree of all nodes in the above directed graph is greater than the set limiting value for the number of the out-degree, and there is no node of which the number of the in-degree is 0 in the above directed graph.

According to a second aspect of the present disclosure, the present disclosure provides a device for generating a DNA storage encoding/decoding rule, includes:
a sliding window setting unit, configured to set a sliding window (n, k) for the DNA storage encoding/decoding rule, wherein n represents a length of the sliding window, and k represents a length of a base character for each sliding, and wherein n and k are positive integers, n≥k;
a qualified sequence screening unit, configured to, based on the length n of the sliding window, obtain a full set of sequences, wherein the full set of the sequences is a set of all base sequences formed by random combination of all base possibilities of each base position within the length of the sliding window, and a set of qualified sequences complying with the above limiting condition in the above full set of the sequences is screened out by using a limiting condition, wherein the limiting condition is set based on a sequence feature in the full set of the sequences;
a directed graph connecting unit, configured to connect the sequences in the above set of the qualified sequences by means of a directed graph, wherein each node in the directed graph represents each sequence;
an out-degree inconsistency deleting unit, configured to delete, in the directed graph, nodes of which a number of out-degree is less than a set limiting value for the number of out-degree;
an excess out-degree deleting unit, configured to delete an excess out-degree of each node in the directed graph, wherein the excess out-degree is an out-degree exceeding the set limiting value for the number of the out-degree; and
an algorithm chart acquiring unit, configured to obtain an algorithm chart, wherein the algorithm chart comprises the DNA storage encoding/decoding rule.

According to a third aspect of the present disclosure, the present disclosure provides a DNA storage encoding method, includes:
the DNA storage encoding/decoding rule generated by the method in the first aspect is obtained, an initial node is set, and the initial node is set as an existing node;
a binary sequence to be encoded is obtained and sliced it to generate a binary slice, and a binary value corresponding to the binary slice is converted into an out-degree node or a multi-layer out-degree node connected with the existing node, wherein each out-degree node describes a nucleic acid fragment, and the above binary slice forms a pair of mapping relationships with a corresponding nucleic acid fragment;
according to the above DNA storage encoding/decoding rule, the above binary slice is input, the nucleic acid fragment mapped by the above out-degree node or the multi-layer out-degree node is output, and the above out-degree node to the existing node is updated, according to an order of the above binary slices, the binary slices and outputting the nucleic acid fragments are continuously and circularly input, until all the above binary slices are input; and
the above nucleic acid fragments sequentially are connected according to an output order and outputting a complete DNA sequence.

Optionally, the above method slices the above binary sequence to be encoded according to a length of 2k−1, wherein k represents a length of a base character for each sliding of a sliding window.

Optionally, the above method further includes: the above DNA sequence is synthesized, and then preserved in a medium in vitro or a living cell.

According to a fourth aspect of the present disclosure, the present disclosure provides a DNA storage encoding device, includes:
an encoding/decoding rule acquiring unit, configured to obtain the DNA storage encoding/decoding rule generated by the method in the first aspect, set an initial node, and set the initial node as an existing node;
a binary sequence slicing and converting unit, configured to obtain a binary sequence to be encoded and slice it to generate a binary slice, and convert a binary value corresponding to the binary slice into an out-degree node or a multi-layer out-degree node connected with the existing node, wherein each out-degree node describes a nucleic acid fragment, and the binary slice forms a pair of mapping relationships with a corresponding nucleic acid fragment;
a nucleic acid fragment outputting unit, configured to, according to the DNA storage encoding/decoding rule, input the binary slice, output a nucleic acid fragment mapped by the out-degree node or the multi-layer out-degree node, and update the out-degree node to the existing node, according to an order of the binary slices, continuously and circularly input the binary slices and output the nucleic acid fragments, until all the binary slices are input; and
a nucleic acid fragment connecting unit, configured to connect the above nucleic acid fragments sequentially according to an output order and output a complete DNA sequence.

Optionally, the above binary sequence to be encoded is sliced according to a length of 2k−1, and wherein k represents a length of a base character for each sliding of a sliding window.

According to a fifth aspect of the present disclosure, the present disclosure provides a DNA storage decoding method, includes:
the DNA storage encoding/decoding rule generated by the method in the first aspect is obtained, an initial node is set, and the initial node is set as an existing node; a DNA sequence to be decoded is obtained and sliced it to generate a nucleic acid slice, and an out-degree node or a multi-layer out-degree node connected with the above existing node is found out according to the above DNA storage encoding/decoding rule and nucleic acid information corresponding to the above nucleic acid slice, wherein each out-degree node describes a piece of the nucleic acid information, and the above nucleic acid slice forms a pair of mapping relationships with a corresponding binary value or binary slice;
according to the above existing node and the above out-degree node or multi-layer out-degree node, the binary value or binary slice between the nodes is obtained according to the above mapping relationship, and the above out-degree node to the existing node is updated, according to an order of the above nucleic acid slices, the nucleic acid slices are continuously and circularly input and the binary value or binary slices are output, until all the above nucleic acid slices are input; and
the above binary value or the binary slices sequentially are connected according to an output order and outputting a complete binary sequence.

Optionally, the above method slices the above DNA sequence to be decoded according to a length of k, wherein k represents a length of a base character for each sliding of a sliding window.

Optionally, the above DNA sequence to be decoded is encoded and generated by the method in the first aspect or the device in the fourth aspect.

According to a sixth aspect of the present disclosure, the present disclosure provides a DNA storage decoding device, includes:
an encoding/decoding rule acquiring unit, configured to obtain the DNA storage encoding/decoding rule generated by the method in the first aspect, set an initial node, and set the initial node as an existing node;
a DNA slicing and converting unit, configured to obtain a DNA sequence to be decoded and slice it to generate a nucleic acid slice, and find out an out-degree node or a multi-layer out-degree node connected with the existing node according to the DNA storage encoding/decoding rule and nucleic acid information corresponding to the nucleic acid slice, wherein each out-degree node describes a piece of the nucleic acid information, and the nucleic acid slice forms a pair of mapping relationships with a corresponding binary value or binary slice;
a binary value outputting unit, configured to, according to the above existing node and the above out-degree node or multi-layer out-degree node, obtain a binary value or a binary slice between the nodes according to the mapping relationship, and update the out-degree node to the existing node, according to an order of the nucleic acid slices, continuously and circularly input the nucleic acid slices and output the binary value or the binary slices, until all the nucleic acid slices are input; and
a binary value connecting unit, configured to connect the above binary value sequentially according to an output order and output a complete binary sequence.

Optionally, the above DNA sequence to be decoded is sliced according to a length of k, wherein k represents a length of a base character for each sliding of a sliding window.

According to a seventh aspect of the present disclosure, the present disclosure provides a computer-readable storage medium, including a program, wherein the program can be executed by a processor to achieve, for example, the method in the first aspect or the method in the third aspect or the method in the fifth aspect.

At present, all the encoding/decoding rules can be generated by the method for generating the DNA storage encoding/decoding rule of the present disclosure, so it is not necessary to set a corresponding encoding/decoding rule for each limiting condition and the encoding efficiency, and the cost is saved.

In addition, based on analysis means of a graph theory, the further theoretical analysis can be performed on the generated implicit encoding/decoding rule, such as the stability analysis of the algorithm. Compared with existing encoding/decoding rules, the encoding/decoding rule generated by the present disclosure has the higher efficiency, because the implicit encoding/decoding rule generated by the present disclosure is an end-to-end direct mapping relationship between binary and base, and the time complexities of encoding and decoding are both O(n). The method of the present disclosure is suitable for sequencing and decoding under any conditions, and can especially be used for sequencing and decoding, while other existing algorithms do not involve the third-generation sequencing and decoding.

### BRIEF DESCRIPTION OF THE DRAWINGS

FIG. 1 is a schematic diagram of an encoding and decoding process of DNA storage in an embodiment of the present disclosure.

FIG. 2 is a flow chart of a method for generating a DNA storage encoding/decoding rule in an embodiment of the present disclosure.

FIG. 3 is a schematic diagram of a method for generating a DNA storage encoding/decoding rule in an embodiment of the present disclosure.

FIG. 4 is a structure block diagram of a device for generating a DNA storage encoding/decoding rule in an embodiment of the present disclosure.

FIG. 5 is a flow chart of a DNA storage encoding method in an embodiment of the present disclosure.

FIG. 6 is a principle schematic diagram of an encoding rule shown in an adjacency matrix or graph mode in a DNA storage encoding/decoding method in an embodiment of the present disclosure.

FIG. 7 is a principle schematic diagram of an encoding/decoding step of a DNA storage encoding/decoding method in an embodiment of the present disclosure.

FIG. 8 is a structure block diagram of a DNA storage encoding device in an embodiment of the present disclosure.

FIG. 9 is a flow chart of a DNA storage decoding method in an embodiment of the present disclosure.

FIG. 10 is a structure block diagram of a DNA storage decoding device in an embodiment of the present disclosure.

FIG. 11 is a schematic diagram of a part of information of a configuration file of an encoding/decoding rule in an embodiment of the present disclosure.

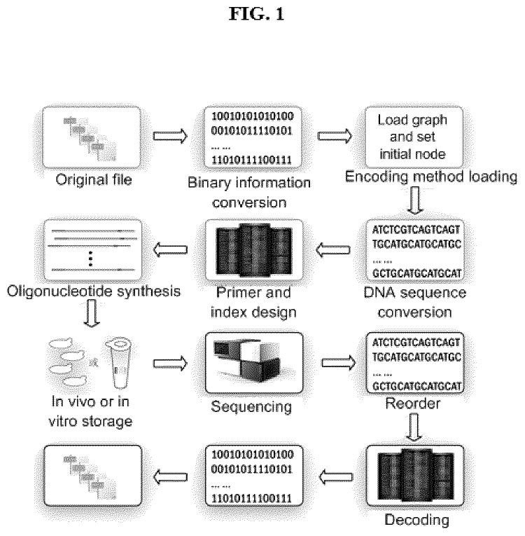

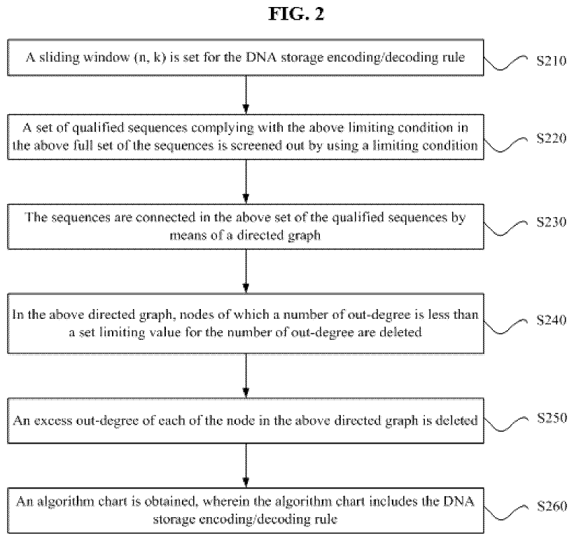

### DETAILED DESCRIPTION OF THE EMBODIMENTS

The present disclosure is further described in detail below by specific embodiments in combination with drawings. In the following embodiments, many details are described so that the present disclosure can be better understood. However, those skilled in the art can easily recognize that some of features can be omitted under different circumstances, or can be replaced by other materials and methods.

In addition, the features, operations or characteristics described in the description can be combined in any suitable manners to form various implementation modes. At the same time, steps or actions in a method description can also be exchanged or adjusted in order in a manner apparent to those skilled in the art. Therefore, various sequences in the description and the drawings are only for clearly describing a certain embodiment, and are not meant to be a necessary order, unless otherwise stated that a certain order must be followed.

### Term Description of the Present Disclosure

Encoding method: refers to a mapping relationship between binary and base. Generally speaking, the traditional encoding method with a fixed rule can perform a plurality of steps of optimization processing, and finally obtain the final mapping relationship. In the present disclosure, the encoding method is achieved by an encoding/decoding rule. The encoding/decoding rule of the present disclosure is generated by a method for generating a DNA storage encoding/decoding rule of the present disclosure.

Generator: also called “the method for generating the DNA storage encoding/decoding rule” in the present disclosure, obtains a certain potential mapping relationship between the binary and the base by means of a method of a graph theory according to different combination situations, namely the encoding/decoding rule of the present disclosure is obtained.

Algorithm stability: means that for arbitrary input electronic files, a DNA sequence output by an algorithm can stably meet a limiting condition. Usually, in the “arbitrary” case, flood-like input can be used to observe the stability of the algorithm in the extreme case.

End-to-end: from original data input to result output, from an input end to an output end, the intermediate mapping processing is self-contained.

Time complexity: the time complexity of an algorithm is a function, and it qualitatively describes the running time of the algorithm. This is a function of the string length representing an input value of the algorithm. The time complexity is often expressed in a big O notation, excluding lower-order term and leading coefficients of the function. While used in this way, the time complexity can be called to be asymptotic, namely the case in which the magnitude of the input value approaches infinity is investigated.

In view of sequence limiting conditions of different sequencing or synthesis instruments, the present disclosure proposes an optimal encoding/decoding generator based on the limiting conditions, namely the method for generating the DNA storage encoding/decoding rule of the present disclosure. This generator (or the method) can solve a problem that existing fixed rules cannot completely avoid extreme GC or special motif. The special motif here refers to a sequence that is difficult to analyze by using the fixed rules.

In addition, the encoding method generated by the generator does not require a screening process, so there is no hidden danger that is unable to accept all inputs. In addition, the encoding/decoding time complexity of the encoding method generated by the generator is O(n). Compared with most encoding/decoding methods that require many optimization processes, the encoding/decoding method of the present disclosure can be much faster, and for large-scale DNA storage transcoding in future, it can be more efficient.

The technical components of the present disclosure are described in detail below, and it should be understood that these descriptions are exemplary, and those skilled in the art can make many modifications on the basis of the technical contents of the present disclosure.

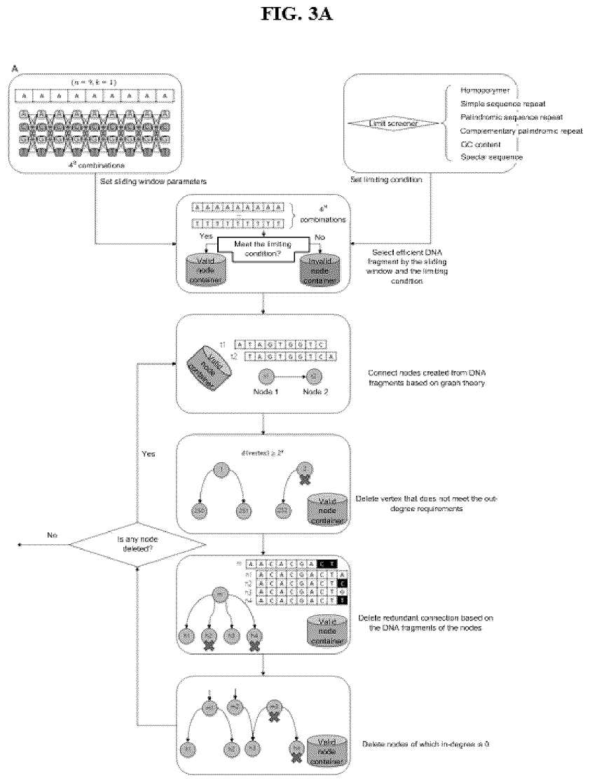

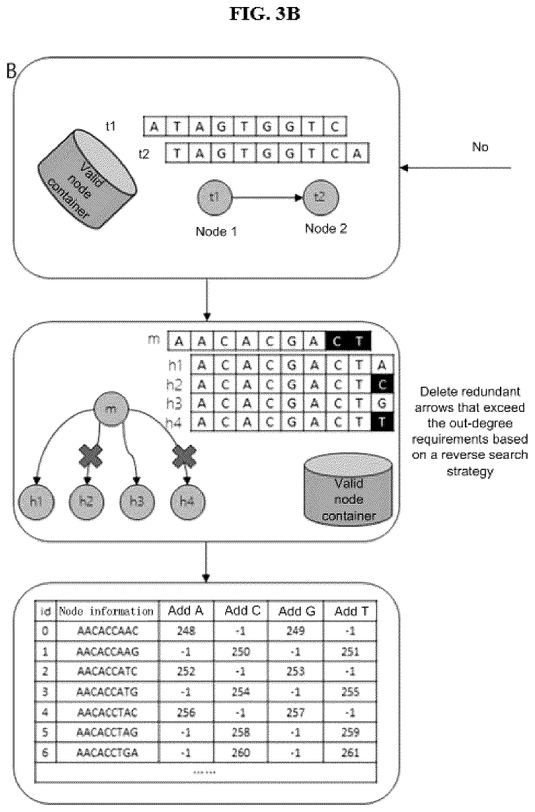

As shown in FIG. 2 and FIG. 3, in an embodiment of the present disclosure, a method for generating a DNA storage encoding/decoding rule, namely a DNA storage encoding/decoding generator, is created based on graph theory and combinatorics, and its steps include:

S210: a sliding window (n, k) is set for the DNA storage encoding/decoding rule.

As shown in FIG. 3, the sliding window is a window model with a fixed length of n. After each sliding of a fixed-length base character k (usually k=1), wherein n and k are positive integers, n≥k, and all character data within the range of the window at an existing position are observed.

S220: based on the length n of the sliding window, a full set of sequences are obtained, wherein the full set of the sequences is a set of all base sequences formed by random combination of all base possibilities of each base position within the length of the sliding window, a set of qualified sequences complying with the above limiting condition in the above full set of the sequences is screened out by using a limiting condition, wherein the limiting condition is set based on a sequence feature in the full set of the sequences.

As shown in FIG. 3, the limiting condition includes at least one of a GC base content, a single base repeat, a simple sequence repeat, a palindromic sequence repeat, a complementary palindromic sequence repeat and a special sequence elimination.

In an embodiment, the limiting condition includes at least one of the followings: the GC base content is 40%-60%, the single base repeat is no more than 3 consecutive identical bases, the simple sequence repeat is not less than 4 bases, the palindromic sequence repeat is not less than 4 bases, the complementary palindromic sequence repeat is not less than 4 bases, and the special sequence elimination is to eliminate a sequence containing AGA, GAG, CTC, and TCT. It should be noted that the “repeat” in the simple sequence repeat, the palindromic sequence repeat and the complementary palindromic sequence repeat refers to the “repeated base length”. For example: a base sequence ACGTACGTACGT(SEQ ID NO: 1), it is a repeat of “ACGT”, and the repeat is 4; and a base sequence ACGTAAACGTAAACGTAA(SEQ ID NO: 2), it is a repeat of “ACGTAA”, and the repeat is 6. Since A base and G base have similar chemical structures, while a third-generation sequencer is used, such as nanopore, for sequencing, the adjacent bases with the similar chemical structures easily cause the base calling confusion during the sequencing process, thereby a sequencing sequence error is caused. Therefore, it is necessary to avoid such sequences as much as possible.

As shown in FIG. 3, a specific operation method for screening out the set of qualified sequences is as follows: (1) since the sequence is composed of a base ACGT, the method of the present disclosure generates 4n sequences (namely the full set of the sequences) in advance; and (2) each sequence is detected by the limiting condition, and if the sequence meets the limiting condition, the sequence is saved in the set of qualified sequences.

S230: the sequences are connected in the above set of the qualified sequences by means of a directed graph, wherein each node in the directed graph represents each sequence.

As shown in FIG. 3, the directed graph is a graph consisting of a number of given nodes and a line connecting two nodes. The directed graph means that the line between the two nodes is directional. Here each sequence is likened to a node in the directed graph. It is assumed that the length of the sequence represented by the node is n, if the character string composed of the 2nd to the n-th characters of the sequence corresponding to a node A is exactly consistent with the character string composed of the 1st to the (n−1)-th characters of the sequence corresponding to another node B, then a connection relationship is from A to B. For example, the length of the sequence represented by the node is 9, a node 1 is a sequence ATAGTGGTC, a node 2 is a sequence TAGTGGTCA, the sequence consisting of the second to the ninth bases of the node 1 sequence is “TAGTGGTC”, and the sequence consisting of the first to the eighth bases of the node 2 sequence is “TAGTGGTC”, the two are exactly the same, and the connection relationship is to be connected from the node 1 to the node 2.

S240: in the above directed graph, nodes of which a number of out-degree is less than a set limiting value for the number of out-degree are deleted.

In an embodiment of the present disclosure, the set limiting value for the number of the out-degree is a number of the out-degree required by an encoding efficiency. As shown in FIG. 3, based on the encoding efficiency, the number of the out-degree of all nodes is checked. If the number of the out-degree of a certain node is less than the number of the out-degree required for the encoding efficiency, the node is deleted. The loop is terminated until all nodes satisfy the condition. The number of the out-degree of the node refers to the number of edges pointing from a given node to other nodes in the directed graph.

In an embodiment, while the encoding efficiency is e, and eϵ(0,2], a limiting value for the number of the out-degree on a k-th (k=e/(2−e)) layer of each node is 2\hat{ }(e/(2−e)). Wherein, k represents the base character length for each sliding of the sliding window, and since the base sequences within the length of one sliding window constitutes the node, the base character length k for each sliding is the k-th layer of the node.

In a more preferred embodiment, while the encoding efficiency is 1, the limit value for the number of the out-degree of each node is 2.

S250: an excess out-degree of each node in the above directed graph is deleted.

In the present disclosure, the excess out-degree is an out-degree exceeding the set limiting value for the number of the out-degree. For example, in an embodiment, for a certain node, if the set limiting value for the number of the out-degree is 2, but the node contains 4 out-degree numbers, then the out-degree exceeding the set limiting value for the number of the out-degree belong to the excess out-degree, namely 2 out-degree need to be deleted. A purpose of deleting the excess out-degree is to maintain the stability of the algorithm.

In an embodiment, the excess out-degree is an out-degree that the total number of the out-degree on the k-th (k=e/(2−e)) layer of the node exceeds 2\hat{ }(e/(2−e)), herein e is the encoding efficiency, and eϵ(0,2].

As shown in FIG. 3, according to the encoding efficiency, the excess out-degree of each node is deleted. A deletion method is specifically as follows: if a total number of the out-degree of the above node exceeds the set limiting value for the number of the out-degree, bases of the above node are output in a reverse order, and the out-degree pointing to a corresponding base sequentially is deleted according to a base order output in the reverse order. In the present disclosure, “the out-degree pointing to the corresponding base” means that in the out-degree formed by the previous node pointing to the next node, if the last base of the base sequence of the next node is the same as the base of the previous node output in the reverse order, the out-degree is “the out-degree pointing to the corresponding base”. For example, the previous node (L) sequence is AACACGACT, the next node sequence connected by this node is respectively as follows: the node (P1) sequence is ACACGACTA, the node (P2) sequence is ACACGACTC, the node (P3) sequence is ACACGACTC and the node (P4) sequence is ACACGACTT, the node (L) is connected with the nodes (P1), (P2), (P3), and (P4) respectively, to form 4 out-degree. If the set limiting value for the number of the out-degree is 2, the number of the excess out-degree is 2, namely 2 excess out-degree need to be deleted, and the bases of the node (L) are output in the reverse order: T, C, A, G, C, A, C, A, A. According to the output order, the out-degree pointing to the T base and the C base are deleted sequentially, namely the out-degree formed by the node (L) and the node (P4) of which the last base sequence is T is deleted, and the out-degree formed by the node (L) and the node (P2) of which the last base sequence is C is deleted.

In some preferred embodiments, after the step S240, further including:

Step S240′: in the above directed graph, nodes of which the number of in-degree is 0 are deleted, as to reduce the range of the directed graph. The advantage of this is that it can improve the strictness degree of the limiting condition on another level for a generating algorithm with a loose limiting condition.

In some preferred embodiments, further includes: after the step S240′ is executed, the step S240 is returned again, and the steps S240-S240′ are executed circularly, until a number of the out-degree of all nodes in the directed graph is greater than the set limiting value for the number of the out-degree, and there is no node of which the number of the in-degree is 0 in the above directed graph.

In some preferred embodiments, between the step S240 and the step S240′, further includes: Step S240″: the excess out-degree of each node in the directed graph is deleted, wherein the excess out-degree is the out-degree exceeding the limiting value for the number of the out-degree. The excess out-degree is defined as above, and is not described repeatedly here.

In some preferred embodiments, further including: after the step (S240′) is deleted, the step (S230) is returned again, and the steps (S230)-(S240)-(S240″)-(S240′) are executed circularly until the number of the out-degree of all nodes in the directed graph is greater than the set limiting value for the number of the out-degree, and there is no node of which the number of the in-degree is 0 in the directed graph. It should be noted that after the end of each cycle, since the case that the node and the out-degree are deleted occurs, while a new cycle starts, all nodes are reconnected according to the above connection principle, then a new directed graph is formed, and the deletion is performed according to the above deletion principle. In addition, by deleting the excess out-degree firstly before deleting the nodes with the number of the in-degree of 0, more nodes with the number of the in-degree of 0 can be exposed in an existing cycle, as to reduce the total number of cycles, and shorten the running time of a program.

S260: an algorithm chart is obtained, wherein the algorithm chart includes the DNA storage encoding/decoding rule.

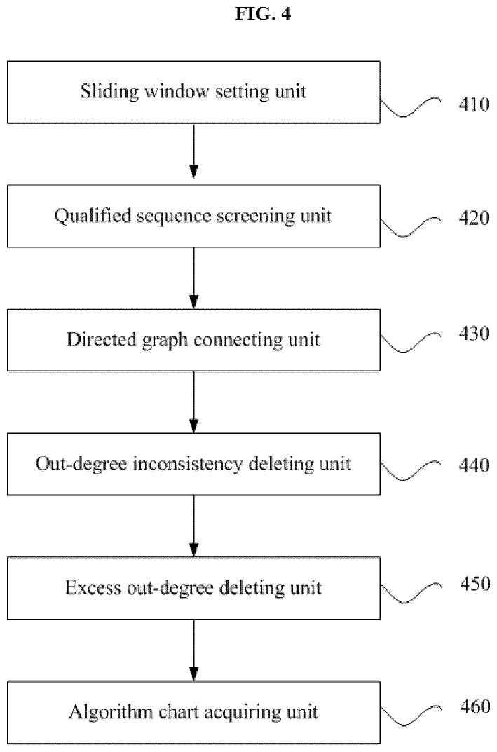

Corresponding to the method for generating the DNA storage encoding/decoding rule of the present disclosure, the present disclosure further provides a device for generating the DNA storage encoding/decoding rule, as shown in FIG. 4, includes: a sliding window setting unit 410, configured to set a sliding window (n, k) for the DNA storage encoding/decoding rule, wherein n represents a length of the sliding window, and k represents a length of a base character for each sliding, and wherein n and k are positive integers, n≥k; a qualified sequence screening unit 420, configured to, based on the length n of the sliding window, obtain a full set of sequences, wherein the full set of the sequences is a set of all base sequences formed by random combination of all base possibilities of each base position within the length of the sliding window, and a set of qualified sequences complying with the above limiting condition in the above full set of the sequences is screened out by using a limiting condition, wherein the limiting condition is set based on a sequence feature in the full set of the sequences; a directed graph connecting unit 430, configured to connect the sequences in the above set of the qualified sequences by means of a directed graph, wherein each node in the directed graph represents each sequence; an out-degree inconsistency deleting unit 440, configured to delete, in the directed graph, nodes of which a number of out-degree is less than a set limiting value for the number of out-degree; an excess out-degree deleting unit 450, configured to delete an excess out-degree of each node in the directed graph, wherein the excess out-degree is an out-degree exceeding the set limiting value for the number of the out-degree; and an algorithm chart acquiring unit 460, configured to obtain an algorithm chart, wherein the algorithm chart comprises the DNA storage encoding/decoding rule.

Those skilled in the art can understand that all or part of functions of the various methods in the above implementation modes can be achieved by means of hardware, or by means of a computer program. While all or part of the functions in the above implementation modes are achieved by means of the computer program, the program can be stored in a computer-readable storage medium, and the storage medium can include: a read-only memory, a random access memory, a magnetic disk, an optical disk, a hard disk and the like. The program is executed by a computer to achieve the above functions. For example, the program is stored in a memory of the device, and while the program in the memory is executed by a processor, all or part of the above functions can be achieved. In addition, while all or part of the functions in the above implementation modes are achieved by means of the computer program, the program can also be stored in a server, another computer, a magnetic disk, an optical disk, a flash disk or a mobile hard disk and other storage media, and saved in a memory of a local device by downloading or copying, or version updating is performed on a system of the local device, while the program in the memory is executed by the processor, all or part of the functions in the above implementation modes can be achieved.

Therefore, an embodiment of the present disclosure provides a computer-readable storage medium, including a program, herein the program can be executed by the processor to achieve the method for generating the DNA storage encoding/decoding rule of the present disclosure.

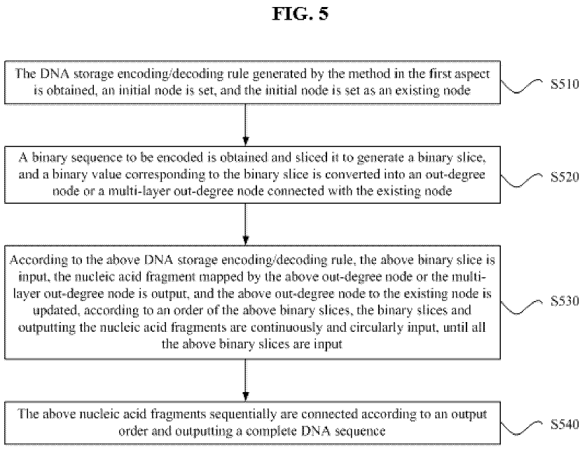

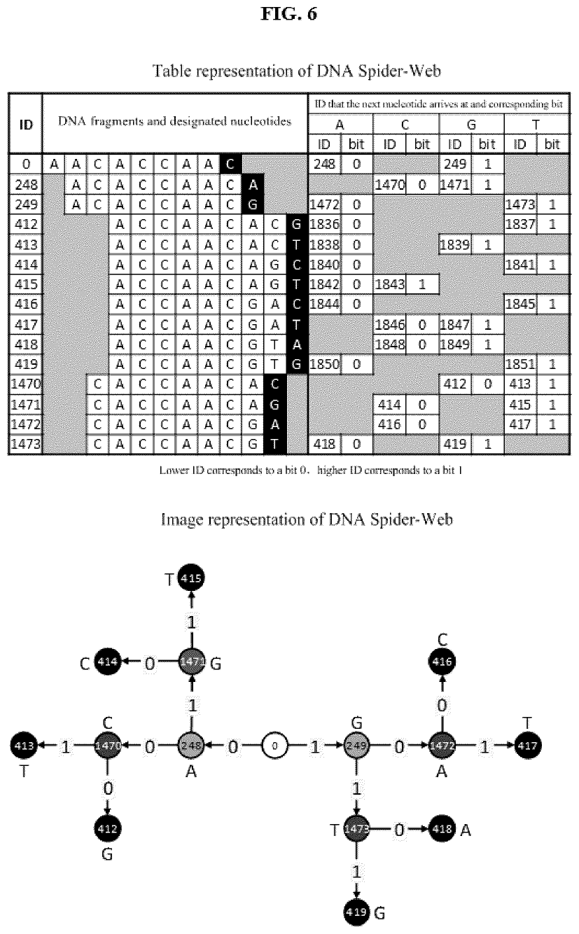

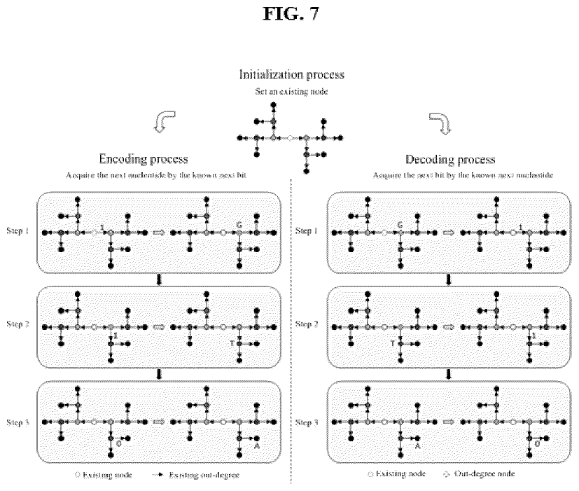

As shown in FIG. 5, FIG. 6, and FIG. 7, an embodiment of the present disclosure further provides a DNA storage encoding method, namely a using method for the generated DNA storage encoding/decoding rule in an encoding phase, includes the following steps:

S510: the DNA storage encoding/decoding rule generated by the method in the first aspect is obtained, an initial node is set, and the initial node is set as an existing node. It can be understood that any node can be set as the initial node, and usually an identity document (ID) of the initial node is set to 0.

Wherein, the DNA storage encoding/decoding rule is generated by the method for generating the DNA storage encoding/decoding rule of the present disclosure under the given sliding window (n, k) and limiting condition.

In an embodiment of the present disclosure, parameters for limiting the sliding window are (n=9, k=1), and the given limiting condition includes: the single base repeat is not exceed 2, the simple sequence repeat is not less than 4 bases, the palindromic sequence repeat is not less than 4 bases, the complementary palindromic sequence repeat is not less than 4 bases, the GC base content is between 40% and 60%, and 4 special sequences “AGA”, “GAG”, “CTC”, and “TCT” for nanopore sequencing are eliminated. In other embodiments, the parameters and limiting condition of the sliding window can be set according to specific needs.

S520: a binary sequence to be encoded is obtained and sliced it to generate a binary slice, and a binary value corresponding to the binary slice is converted into an out-degree node or a multi-layer out-degree node connected with the existing node, wherein each out-degree node describes a nucleic acid fragment, and the above binary slice forms a pair of mapping relationships with a corresponding nucleic acid fragment.

In an embodiment of the present disclosure, the above binary sequence to be encoded according to a length of 2k−1, wherein k represents a length of a base character for each sliding of a sliding window.

S530: according to the above DNA storage encoding/decoding rule, the above binary slice is input, the nucleic acid fragment mapped by the above out-degree node or the multi-layer out-degree node is output, and the above out-degree node to the existing node is updated, according to an order of the above binary slices, the binary slices and outputting the nucleic acid fragments are continuously and circularly input, until all the above binary slices are input.

In an embodiment of the present disclosure, an adjacency matrix is used to display the principle of the encoding method, as shown in FIG. 6. In the adjacency matrix, white text on a black background represents a specified nucleotide under the existing ID. In the figure, the color of the node from light to dark represents the number of layers corresponding to the node, the number in the node represents ID of the node, and a character closest to the node represents the specified nucleotide in the node. Each arrow represents a bit obtained from this node to the next node.

In an embodiment of the present disclosure, an adjacency matrix graph (DNA Spider-Web) is used to display the specific encoding and decoding process, as shown in FIG. 7. The figure shows a process of jumping to the next node from the node in the figure after reading a bit in the encoding process, and a process of acquiring the corresponding nucleotide in this process.

S540: the above nucleic acid fragments sequentially are connected according to an output order and outputting a complete DNA sequence.

In an embodiment of the present disclosure, in the DNA storage encoding method of the present disclosure, after the complete DNA sequence is output, the above DNA sequence is synthesized, and then preserved in a medium in vitro or a living cell.

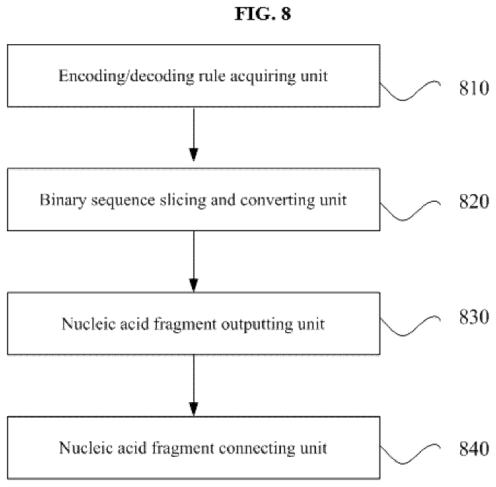

Corresponding to the DNA storage encoding method of the present disclosure, an embodiment of the present disclosure further provides a DNA storage encoding device, as shown in FIG. 8, includes: an encoding/decoding rule acquiring unit 810, configured to obtain the DNA storage encoding/decoding rule generated by the method for generating the DNA storage encoding/decoding rule in the present disclosure, set an initial node, and set the initial node as an existing node; a binary sequence slicing and converting unit 820, configured to obtain a binary sequence to be encoded and slice it to generate a binary slice, and convert a binary value corresponding to the binary slice into an out-degree node or a multi-layer out-degree node connected with the existing node, wherein each out-degree node describes a nucleic acid fragment, and the binary slice forms a pair of mapping relationships with a corresponding nucleic acid fragment; a nucleic acid fragment outputting unit 830, configured to, according to the above DNA storage encoding/decoding rule, input the above binary slice, output a nucleic acid fragment mapped by the out-degree node or the multi-layer out-degree node, and update the out-degree node to the existing node, according to an order of the binary slices, continuously and circularly input the binary slices and output the nucleic acid fragments, until all the binary slices are input; and a nucleic acid fragment connecting unit 840, configured to connect the above nucleic acid fragments sequentially according to an output order and output a complete DNA sequence.

In addition, an embodiment of the present disclosure provides a computer-readable storage medium, including a program, herein the program can be executed by the processor to achieve the DNA storage encoding method of the present disclosure.

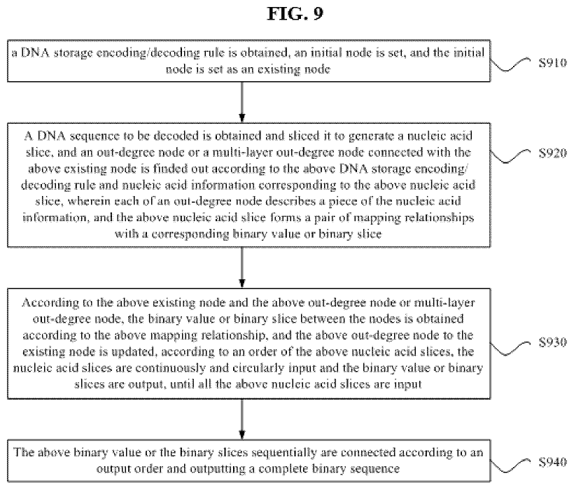

As shown in FIG. 7 and FIG. 9, an embodiment of the present disclosure further provides a DNA storage decoding method, namely a using method for the generated DNA storage encoding/decoding rule in a decoding phase, includes the following steps:

S910: a DNA storage encoding/decoding rule is obtained, an initial node is set, and the initial node is set as an existing node. It can be understood that any node can be set as the initial node, and usually ID of the initial node is set to 0.

Wherein, the DNA storage encoding/decoding rule is generated by the method for generating the DNA storage encoding/decoding rule of the present disclosure under the given sliding window (n, k) and limiting condition.

In an embodiment of the present disclosure, parameters for limiting the sliding window are (n=9, k=1), and the given limiting condition includes: the single base repeat is not exceed 2, the simple sequence repeat is not less than 4 bases, the palindromic sequence repeat is not less than 4 bases, the complementary palindromic sequence repeat is not less than 4 bases, the GC base content is between 40% and 60%, and 4 special sequences “AGA”, “GAG”, “CTC”, and “TCT” for nanopore sequencing are eliminated. In other embodiments, the parameters and limiting condition of the sliding window can be set according to specific needs.

S920: a DNA sequence to be decoded is obtained and sliced it to generate a nucleic acid slice, and an out-degree node or a multi-layer out-degree node connected with the above existing node is found out according to the above DNA storage encoding/decoding rule and nucleic acid information corresponding to the above nucleic acid slice, wherein each out-degree node describes a piece of the nucleic acid information, and the above nucleic acid slice forms a pair of mapping relationships with a corresponding binary value or binary slice.

In an embodiment of the present disclosure, the DNA sequence to be decoded is sliced according to a length of k, wherein k represents a length of a base character for each sliding of a sliding window.

S930: according to the above existing node and the above out-degree node or multi-layer out-degree node, the binary value or binary slice between the nodes is obtained according to the above mapping relationship, and the above out-degree node to the existing node is updated, according to an order of the above nucleic acid slices, the nucleic acid slices are continuously and circularly input and the binary value or binary slices are output, until all the above nucleic acid slices are input.

In an embodiment of the present disclosure, an adjacency matrix graph (DNA Spider-Web) is used to display the specific encoding and decoding process, as shown in FIG. 7. The figure shows a process of jumping to the next node from the node in the figure after reading a nucleotide in the decoding process, and a process of acquiring the corresponding bit in this process.

S940: the above binary value or the binary slices sequentially are connected according to an output order and outputting a complete binary sequence.

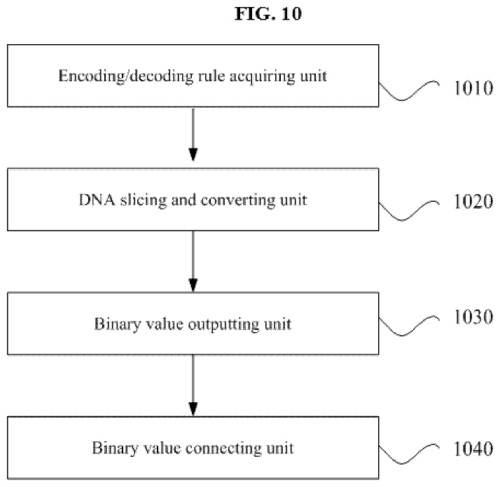

Corresponding to the DNA storage decoding method of the present disclosure, an embodiment of the present disclosure further provides a DNA storage decoding device, as shown in FIG. 10, includes: an encoding/decoding rule acquiring unit 1010, configured to obtain the DNA storage encoding/decoding rule generated by the method for generating the DNA storage encoding/decoding rule in the present disclosure, set an initial node, and set the initial node as an existing node; a DNA slicing and converting unit 1020, configured to obtain a DNA sequence to be decoded and slice it to generate a nucleic acid slice, and find out an out-degree node or a multi-layer out-degree node connected with the above existing node according to the above DNA storage encoding/decoding rule and nucleic acid information corresponding to the above nucleic acid slice, wherein each out-degree node describes a piece of the nucleic acid information, and the above nucleic acid slice forms a pair of mapping relationships with a corresponding binary value or binary slice; a binary value outputting unit 1030, configured to, according to the above existing node and the above out-degree node or multi-layer out-degree node, obtain a binary value or a binary slice between the nodes according to the above mapping relationship, and update the above out-degree node to the existing node, according to an order of the above nucleic acid slices, continuously and circularly input the nucleic acid slices and output the binary value or binary slices, until all the above nucleic acid slices are input; and a binary value connecting unit 1040, configured to connect the above binary value or binary slices sequentially according to an output order and output a complete binary sequence.

In addition, an embodiment of the present disclosure provides a computer-readable storage medium, including a program, herein the program can be executed by the processor to achieve the DNA storage decoding method of the present disclosure.

The technical schemes and effects of the present disclosure are described in detail below by the embodiments. It should be understood that the embodiments are only exemplary, and should not be construed as limitation to the present disclosure.

In this embodiment, the parameters for limiting the sliding window are (n=9, k=1).

The process of acquiring the implicit encoding/decoding rule by the DNA storage encoding/decoding generator is as follows:
(1) Based on the existing limiting condition (the single base repeat is not exceed 2, the simple sequence repeat is not less than 4 bases, the palindromic sequence repeat is not less than 4 bases, the complementary palindromic sequence repeat is not less than 4 bases, the GC content is between 40% and 60%, and 4 special sequences “AGA”, “GAG”, “CTC”, and “TCT” for nanopore sequencing are eliminated), all combination cases are obtained. In original 4n=262144 DNA sequence fragment combinations, the DNA sequence fragments that do not meet the above limiting condition are ignored, the remaining DNA sequence fragments are retained, and finally the set of qualified sequences including 48460 combination modes is obtained.
(2) The sequences in the set of qualified sequences are connected in a mode of the directed graph. The out-degree condition of each node is detected, and nodes that do not meet the requirements are deleted, namely the nodes of which the out-degree is not exceed 2k=2, until all the remaining nodes meet the requirements. After a total of 9 rounds of the screening process, 14,000 combination modes are finally obtained.
(3) Nodes of which in-degree is 0 are eliminated, and further the out-degree condition is detected. After a total of 10 in-degree elimination operations, 5264 DNA sequence fragment combinations are finally obtained.
(4) In the 5264 DNA sequence fragment combinations, all nodes of which the out-degree is greater than 2 are found out. The sequences of these nodes are output in the reverse order. If there are the excess out-degree, the out-degree pointing to the corresponding bases are deleted sequentially according to an order of the sequences in the reverse order. The adjacency matrix of the graph is saved, and the encoding/decoding method including the implicit encoding/decoding rule is generated.

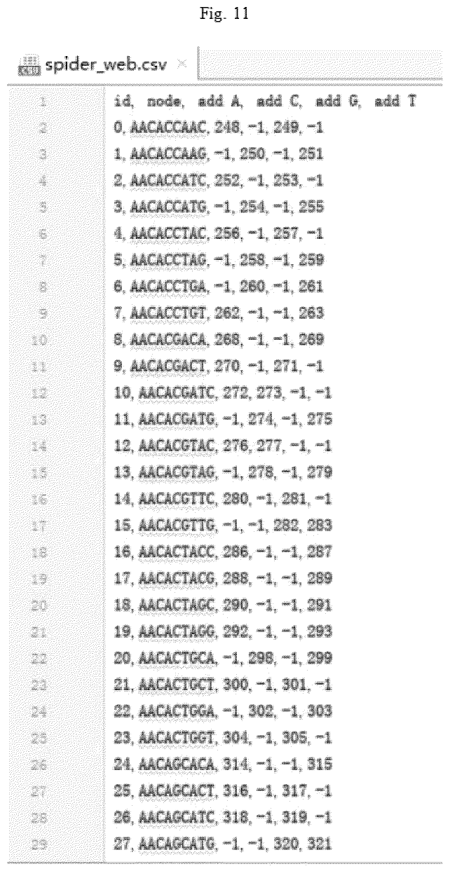

A specific using embodiment of the generated encoding/decoding method in encoding and decoding is as follows:
(1) Precondition: the encoding/decoding method including the implicit encoding/decoding rule generated in this embodiment is obtained, and a part of information of a configuration file of the encoding/decoding rule is shown in FIG. 11.
(2) Specific storage process:
[1] A binary code corresponding to this sentence “Hello world!” is extracted:
0001001010100110001101100011011011110110000001001110111011110110010011100011 01100010011010000100.
[2] The above binary code is sliced according to the length of 1, and binary information is encoded according to the encoding rule, to obtain DNA fragments, and each DNA fragment is connected according to an order of slices, to obtain the following full-length DNA sequence:

(SEQ ID NO: 3)
ACGTACCACATCAGTCACGTAGTCAGTGCTGATGTGCTGACAACCTACG
TTCGTGATGTGCTGACTACGTTCACGTAGTCAGTCATGCTACACTAC.

[3] The above full-length DNA sequence is synthesized by using a method of chemical synthesis.
[4] Synthesized DNA molecules are saved, to achieve the information storage.
(3) Specific reading process:
[1] A specific sequence of the stored DNA molecule is obtained by sequencing, as follows:

(SEQ ID NO: 3)
ACGTACCACATCAGTCACGTAGTCAGTGCTGATGTGCTGACAACCTACG
TTCGTGATGTGCTGACTACGTTCACGTAGTCAGTCATGCTACACTAC.

[2] The above DNA sequence is sliced according to the length of 1, and sequence information is decoded according to the decoding rule, to obtain binary values, and each binary value is connected in an order of slices, to obtain the following binary sequence:
0001001010100110001101100011011011110110000001001110111011110110010011100011 01100010011010000100.
[3] The binary information is restored, and interpreted as Hello world!.

The specific examples are used to describe the present disclosure above, are only used to help understand the present disclosure, and are not intended to limit the present disclosure. For those skilled in the art to which the present disclosure belongs, according to the idea of the present disclosure, several simple deductions, modifications or substitutions can also be made.

1. A method for generating a DNA storage encoding/decoding rule, comprising:
(1) setting a sliding window (n, k) for the DNA storage encoding/decoding rule, wherein n represents a length of the sliding window, and k represents a length of a base character for each sliding, and wherein n and k are positive integers, n≥k;
(2) based on the length n of the sliding window, obtaining a full set of sequences, wherein the full set of the sequences is a set of all base sequences formed by random combination of all base possibilities of each base position within the length of the sliding window, using a limiting condition, to screen out a set of qualified sequences complying with the limiting condition in the full set of the sequences, wherein the limiting condition is set based on a sequence feature in the full set of the sequences;
(3) connecting the sequences in the set of the qualified sequences by means of a directed graph, wherein each node in the directed graph represents each sequence;
(4) deleting, in the directed graph, nodes of which a number of out-degree is less than a set limiting value for the number of the out-degree;
(5) deleting an excess out-degree of each node in the directed graph, wherein the excess out-degree is an out-degree exceeding the set limiting value for the number of the out-degree; and
(6) obtaining an algorithm chart, wherein the algorithm chart comprises the DNA storage encoding/decoding rule.

2. The method according to claim 1, wherein the limiting condition comprises at least one of a GC base content, a single base repeat, a simple sequence repeat, a palindromic sequence repeat, a complementary palindromic sequence repeat and a special sequence elimination.

3. The method according to claim 2, wherein the limiting condition comprises at least one of the followings:
the GC base content is 40%-60%, the single base repeat is no more than 3 consecutive identical bases, the simple sequence repeat is not less than 4 bases, the palindromic sequence repeat is not less than 4 bases, the complementary palindromic sequence repeat is not less than 4 bases, and the special sequence elimination is to eliminate a sequence containing AGA, GAG, CTC, and TCT.

4. The method according to claim 1, wherein the set limiting value for the number of the out-degree is a number of the out-degree required by an encoding efficiency.

5. The method according to claim 4, wherein the encoding efficiency is e, and while eϵ(0,2], a limiting value for the number of the out-degree on a k-th (k=e/(2−e)) layer of each node is 2\hat{ }(e/(2−e)).

6. The method according to claim 5, wherein while the encoding efficiency is 1, the limiting value for the number of the out-degree of each node is 2.

7. The method according to claim 1, wherein the step of deleting the excess out-degree of each node in the directed graph comprises: if a total number of the out-degree of the node exceeds the set limiting value for the number of the out-degree, outputting bases of the node in a reverse order, and deleting an out-degree pointing to a corresponding base sequentially according to a base order output in the reverse order.

8. The method according to claim 1, wherein after the step (4), the method further comprises:
(4′) deleting, in the directed graph, nodes of which a number of in-degree is 0.

9. The method according to claim 8, wherein the method further comprises:
after executing the step (4′), returning to the step (4) again, and executing the steps (4)-(4′) circularly until a number of the out-degree of all nodes in the directed graph is greater than the set limiting value for the number of the out-degree, and there is no node of which the number of the in-degree is 0 in the directed graph.

10. The method according to claim 8, wherein between the step (4) and the step (4′), the method further comprises:
(4″) deleting the excess out-degree of each node in the directed graph, wherein the excess out-degree is the out-degree exceeding the limiting value for the number of the out-degree.

11. The method according to claim 10, wherein the method further comprises:
after executing the step (4′), returning to the step (3) again, and executing the steps (3)-(4)-(4″)-(4′) circularly until the number of the out-degree of all nodes in the directed graph is greater than the set limiting value for the number of the out-degree, and there is no node of which the number of the in-degree is 0 in the directed graph.

12. (canceled)

13. A DNA storage encoding method, wherein the method comprises:
obtaining the DNA storage encoding/decoding rule generated by the method according to claim 1, setting an initial node, and setting the initial node as an existing node;
obtaining a binary sequence to be encoded and slicing it to generate a binary slice, and converting a binary value corresponding to the binary slice into an out-degree node or a multi-layer out-degree node connected with the existing node, wherein each out-degree node describes a nucleic acid fragment, and the binary slice forms a pair of mapping relationships with a corresponding nucleic acid fragment;
according to the DNA storage encoding/decoding rule, inputting the binary slice, outputting a nucleic acid fragment mapped by the out-degree node or the multi-layer out-degree node, and updating the out-degree node to the existing node, according to an order of the binary slices, continuously and circularly inputting the binary slices and outputting the nucleic acid fragments, until all the binary slices are input; and
connecting the nucleic acid fragments sequentially according to an output order and outputting a complete DNA sequence.

14. The method according to claim 13, wherein the method slices the binary sequence to be encoded according to a length of 2k−1, wherein k represents a length of a base character for each sliding of a sliding window.

15. The method according to claim 13, wherein the method further comprises: synthesizing the DNA sequence, and then preserving in a medium in vitro or a living cell.

16. (canceled)

17. (canceled)

18. A DNA storage decoding method, wherein the method comprises:
obtaining the DNA storage encoding/decoding rule generated by the method according to claim 1, setting an initial node, and setting the initial node as an existing node;
obtaining a DNA sequence to be decoded and slicing it to generate a nucleic acid slice, and finding out an out-degree node or a multi-layer out-degree node connected with the existing node according to the DNA storage encoding/decoding rule and nucleic acid information corresponding to the nucleic acid slice, wherein each out-degree node describes a piece of the nucleic acid information, and the nucleic acid slice forms a pair of mapping relationships with a corresponding binary value or binary slice;
according to the existing node and the out-degree node or multi-layer out-degree node, obtaining a binary value or a binary slice between the nodes according to the mapping relationships, and updating the out-degree node to the existing node, according to an order of the nucleic acid slices, continuously and circularly inputting the nucleic acid slices and outputting the binary value or the binary slices, until all the nucleic acid slices are input; and
connecting the binary value or the binary slices sequentially according to an output order and outputting a complete binary sequence.

19. The method according to claim 18, wherein the method slices the DNA sequence to be decoded according to a length of k, wherein k represents a length of a base character for each sliding of a sliding window.

20. The method according to claim 18, wherein the DNA sequence to be decoded is encoded and generated by the method according to claim 13.

21. (canceled)

22. (canceled)

23. (canceled)

24. The method according to claim 9, wherein between the step (4) and the step (4′), the method further comprises:
(4″) deleting the excess out-degree of each node in the directed graph, wherein the excess out-degree is the out-degree exceeding the limiting value for the number of the out-degree.

25. The method according to claim 18, wherein the DNA sequence to be decoded is encoded and generated by the method according to claim 14.
